(19) United States
(12) Patent Application Publication
(10) Pub. No.: US 2014/0142576 A1
(43) Pub. Date: May 22, 2014

(54) STERNUM FIXATION SYSTEM

(71) Applicant: James R. Shipley, San Antonio, TX (US)

(72) Inventor: James R. Shipley, San Antonio, TX (US)

(21) Appl. No.: 14/083,547

(22) Filed: Nov. 19, 2013

Related U.S. Application Data
(60) Provisional application No. 61/728,351, filed on Nov. 20, 2012.

(51) Int. Cl. A61B 17/82

(52) U.S. Cl. CPC A61B 17/823
USPC 606/74

(57) ABSTRACT

A sternum fixation assembly is disclosed for fixing the sternum following a modified median sternotomy. The sternotomy divides a median into left and right median facing walls. A pair of left and right opposing excavations are made in the walls. The assembly comprises an implantable bio-absorbable transverse member configured for enclosure into the excavations of the left and right halves of the vertically divided sternum. An elongated coupleable adjustable compression member is dimensioned for receipt about the inter-rib spaces about the sternum and adapted to apply compression to the outer ridges of the sternum to help maintain the sternum enclosure.

This application claims priority to and the benefit of U.S. provisional patent application Ser. No. 61/728,352, filed Nov. 20, 2012.

### FIELD OF THE INVENTION

A sternum fixation system, more specifically, an alignment and fixation system for fixing or reducing the sternum following a median sternotomy or similar operation.

### BACKGROUND OF THE INVENTION

The sternum is the flat bone in the middle of the chest that holds the ribs together. A median sternotomy is a surgical procedure in which a vertical incision is made along the sternum, after which the sternum is divided or “cracked.” This procedure provides access to the heart and lungs for surgical procedures, such as a heart transplant, corrective surgery for congenital heart defects or coronary artery bypass surgery. In a median sternotomy, the sternum is typically divided vertically into two approximately equal parts, which will be approximated, reduced or joined at the end of the operation, typically to be held together with steel wires.

There are a variety of sternotomy closure techniques. Some use external plates and screws. Some, such as the Synthes® Sternal Zip Fix System, uses PEEK (polyether ether ketone) implants, similar to cable-ties or zip ties.

### SUMMARY OF THE INVENTION

A sternum fixation system for fixing the sternum following a modified median sternotomy is disclosed. A vertically divided sternum has left and right median facing walls created by the sternotomy, which include paired left and right opposing excavations. The system includes an implantable, transverse, bio-absorbable member configured for enclosure into the excavations of the left half and a right half of a vertically divided sternum; and an elongated, coupleable, adjustable one-way member dimensioned for receipt about the inter-rib spaces about the sternum and adapted to apply tension to the outer ridges of the sternum to maintain the sternum in closure.

### BRIEF DESCRIPTION OF THE DRAWINGS

FIG. 1 is a perspective view of Applicant's sternum fixation system with the internal transverse members and the adjustable compression members in place and the sternum closed.

FIG. 2 is an exploded perspective view of one embodiment of Applicant's sternum fixation system.

FIGS. 3, 3A, 3B, and 3C are perspective views of four transverse members for use in Applicant's sternum fixation system.

FIG. 4 is a perspective view of an alternate embodiment of Applicant's sternum fixation system.

FIG. 5 is an exploded view of the system in FIG. 4.

FIG. 6 is an external perspective view showing the use of “one-way” tightening compression members, dimensioned for receipt about the inter-rib spaces about the sternum.

FIG. 7 is a perspective view of a compression member.

FIG. 7A illustrates in side cross-sectional view, the manner in which a barbed embodiment, when inserted into a hole, makes contact between the hole walls, which causes the barbs to flex inward.

### DETAILED DESCRIPTION OF THE PREFERRED EMBODIMENTS

Applicant provides a system for maintaining closure and reducing flex following a median sternotomy. More specifically, when a sternum is cut vertically and pulled apart for heart and lung operations, for example, Applicant provides a modification to the reduction or closure of the sternum halves following the sternotomy, which modification provides for the surgeon to excavate paired holes in both opposed faces generated by the sternotomy. Following such excavation, transverse elements or members are placed in the holes and the opposed faces are brought together and held in place with compression members.

Applicant provides an implantable, transverse, typically bio-absorbable, longitudinal or disk-shaped member for placement and enclosure into the excavations on both faces, such that when the two halves are closed, the implantable transverse alignment members cause the two halves to resist displacement or shifting out of the planar alignment approximated by the closure. Moreover, Applicant provides adjustable closure compression members, typically one-way members, in which compression may be maintained to keep the faces in closure during the healing process.

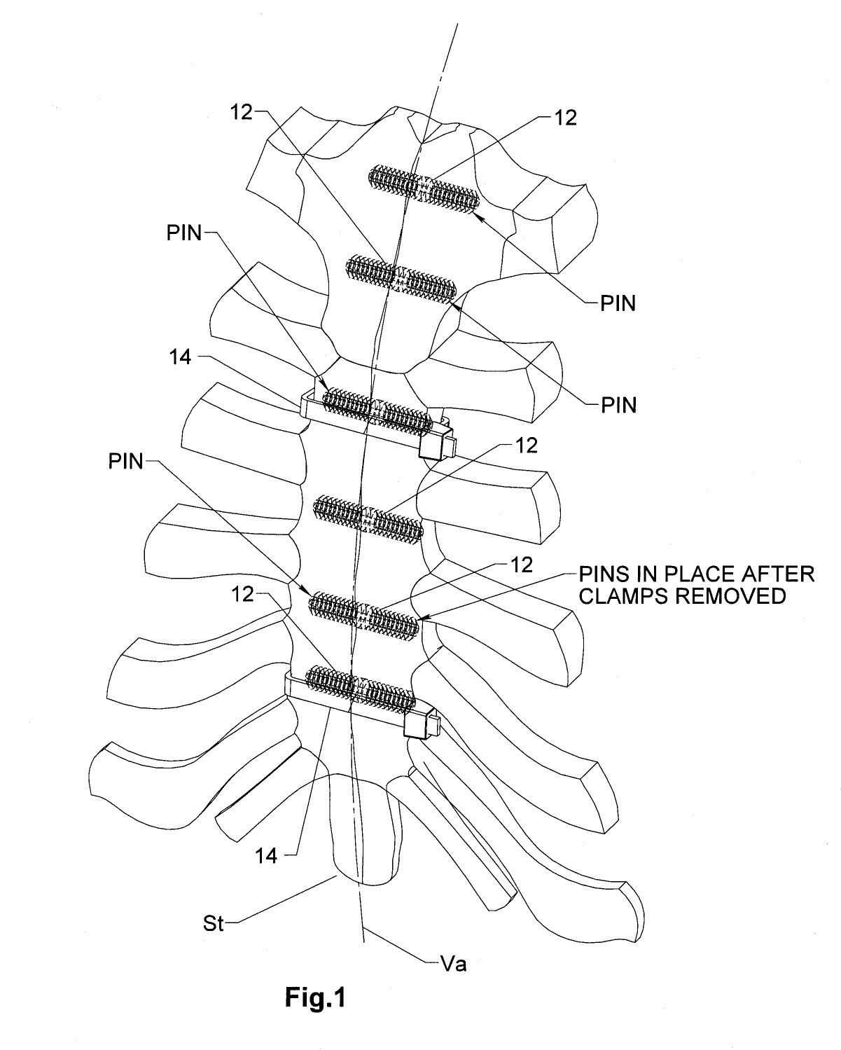

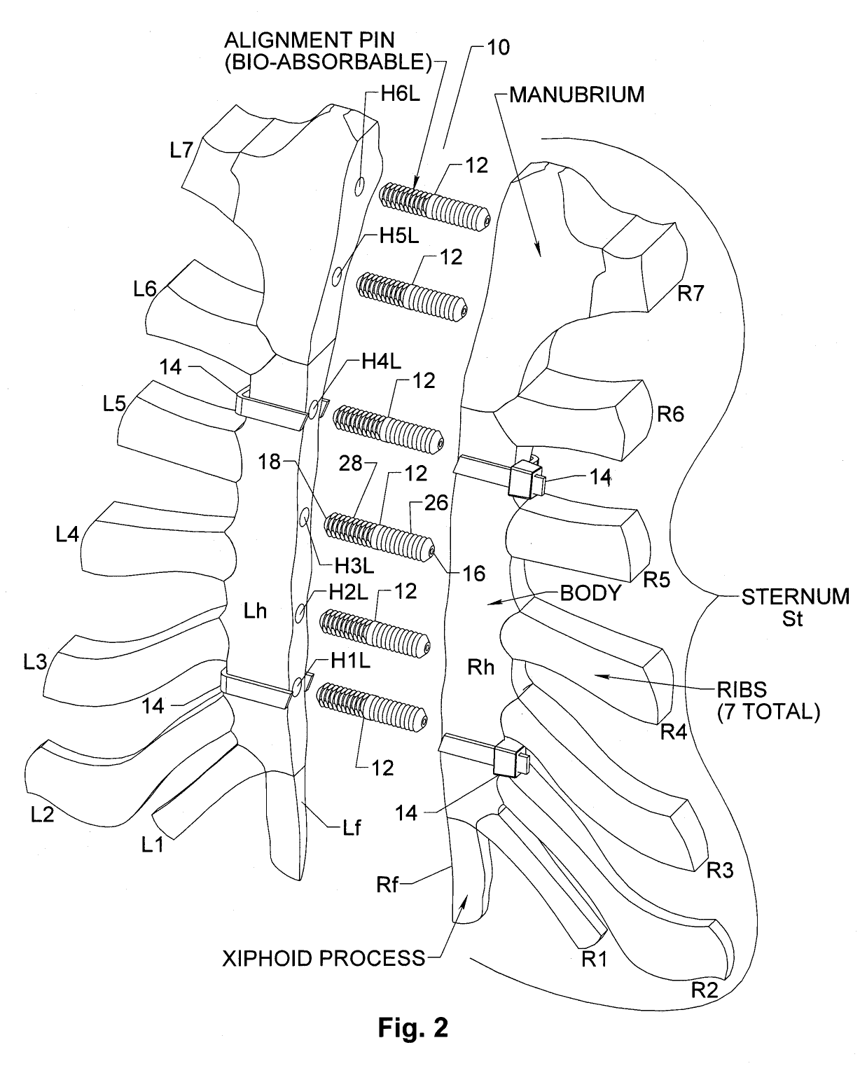

Turning now to FIGS. 1-6, it is seen that Applicant provides a sternum fixation system 10 for use in a modified median sternotomy, in which a sternum St is divided along a vertical axis Va into a left half Lh and a right half Rh. Seven ribs R1-R7/L1-L7 attached to the sternum on the left and right are seen in the Figures. Up to six paired holes H1L-H6L and H1R-H6R are provided, which are excavated by the surgeon to conform to the shape of an internal transverse alignment member as set forth more hereinbelow.

Applicant's sternum fixation system 10 includes internal alignment members in a number of embodiments, which are adapted with walls configured to the holes and dimensioned for receipt into the paired holes such that, upon closure, part of the internal alignment member is in the left half and part is in the right half of the joined sternum. An external compression member 14, generically shown, provides compression and is placed between the two faces Lf and Rf against one another for healing following the modified medium sternotomy.

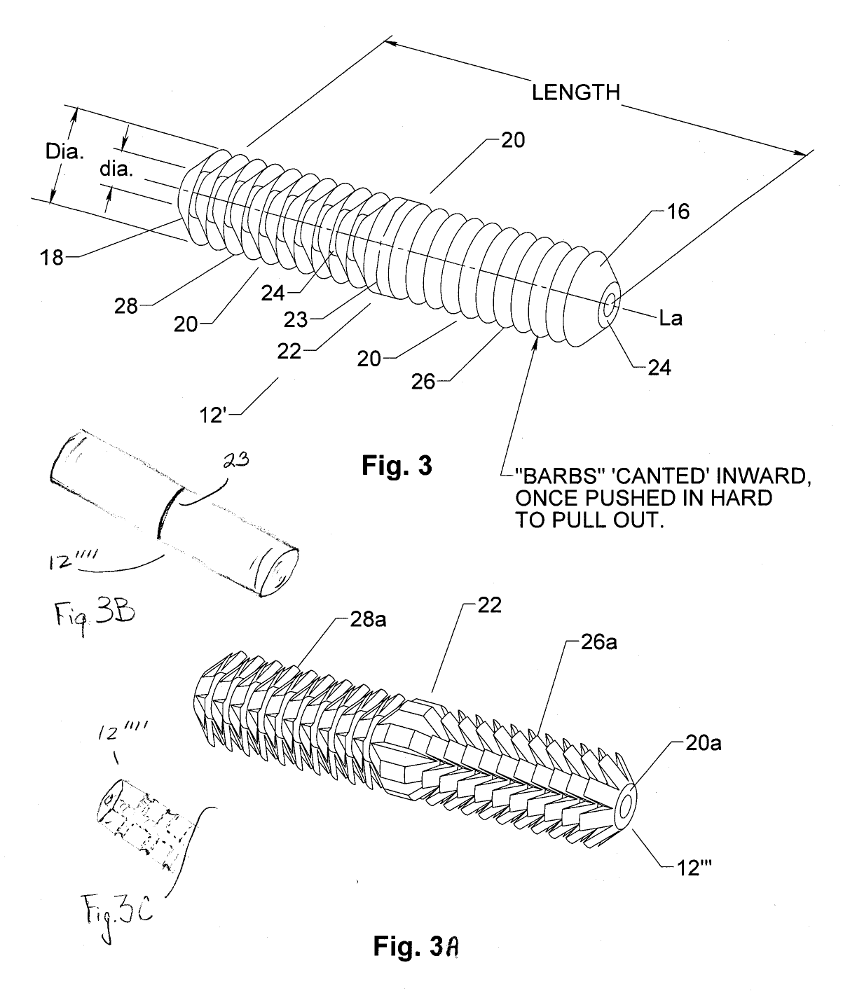

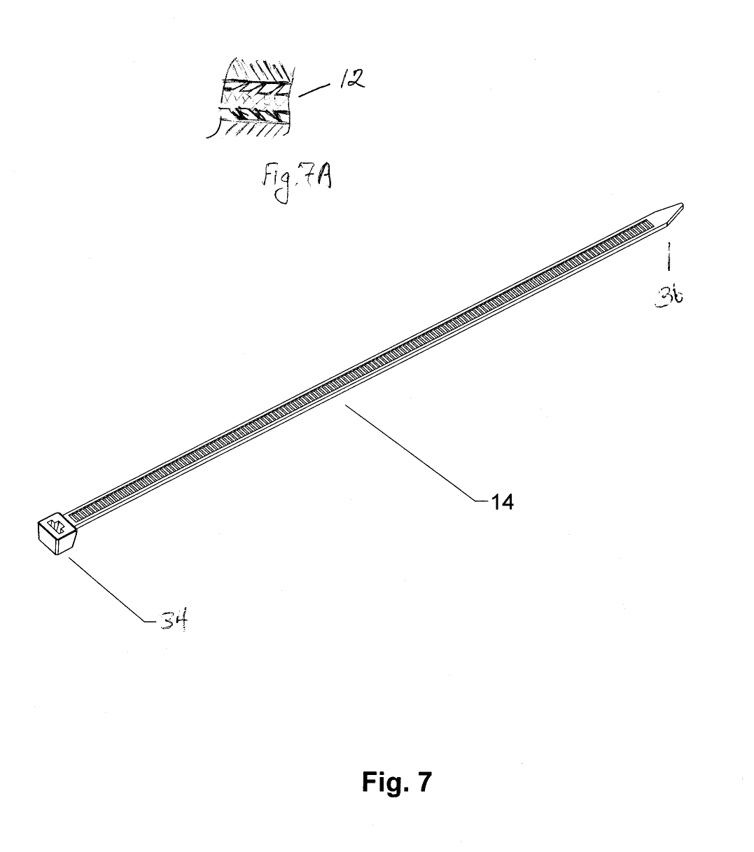

Turning to FIGS. 1, 2, and 3, it is seen that Applicant provides in one embodiment an alignment pin 12′ having a longitudinal axis La and first ends and second ends 16/18. In this embodiment, alignment pin 12′ is seen to comprise a generally cylindrical elongated member, wherein the shape of the excavated holes are adapted to conform to the shape of alignment pin 12′ for snug receipt of the alignment pins into the paired hole sets as seen in FIG. 1, for example. First and second ends 16/18 may be straight, tapered or cone shaped for easy insertion into their respective holes in the sternum halves as set forth herein. Moreover, in one embodiment, alignment pin 12′ has a body 20 and a central portion 22, which may be lined or marked 23 to indicate that it is centrally located between the removed ends for the surgeon performing the closure operation to know when the body half is fully inserted into the excavated hole portions. Length L of alignment pin 12′ and diameter Dia of the body may vary and would be selected to be suitable to the size and age of the patient. Body 20, in one embodiment 12′ (see FIG. 3), may be solid and comprised of a core 24 with a multiplicity of inwardly canted full perimeter barb bodies 26/28 thereupon. The barbs are typically angled inwards towards center portion 22. When placed in the sternum holes, the barbs contact the walls and flex inward slightly (see FIG. 7A) Moreover, alignment pin 12′ indeed the other embodiments of the alignment members may be made of a flexible, pliable or elastomeric material or at least the barb bodies 26/28 of alignment pin 12′ may be made of such a material. With such an elastomeric material comprising barb bodies 26/28, along with their inward cant, it will be seen that they may be more easily pushed into the excavated holes than drawn out. Like arrowheads, barbs tend to go one way more easily than their reverse direction.

The alignment members disclosed herein may be made of bio-absorbable, flexible, pliable or elastomeric materials or any other suitable material. One bioabsorbable material is made by MTD Micromolding, Charlton, Mass. (see www.mtdmicromolding.com). Another is a PLGA material (lactic/glycolic acid co-polymer).

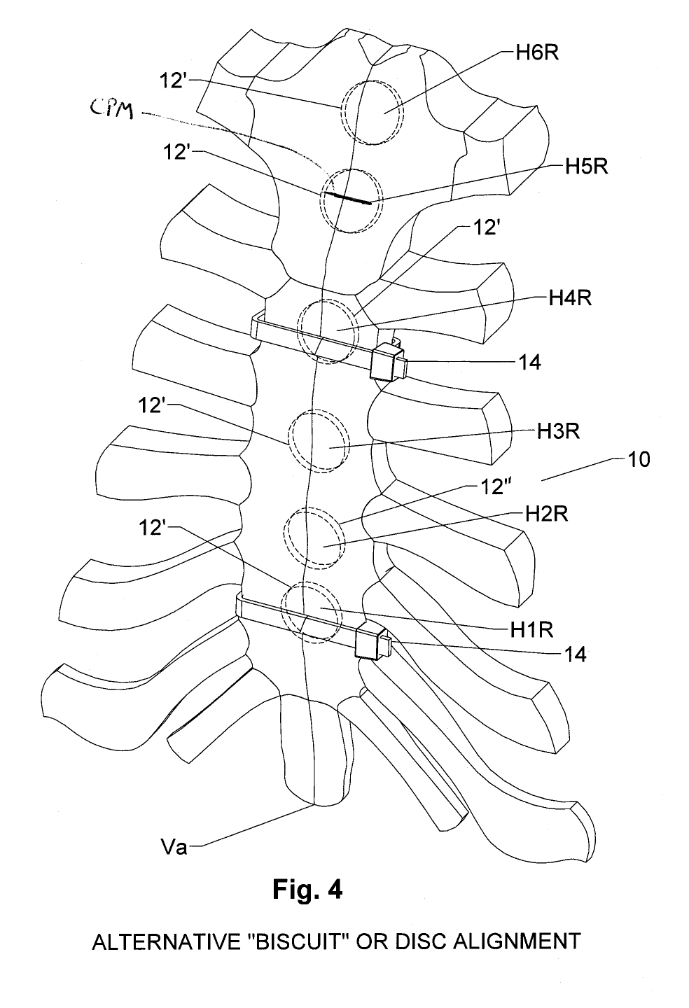

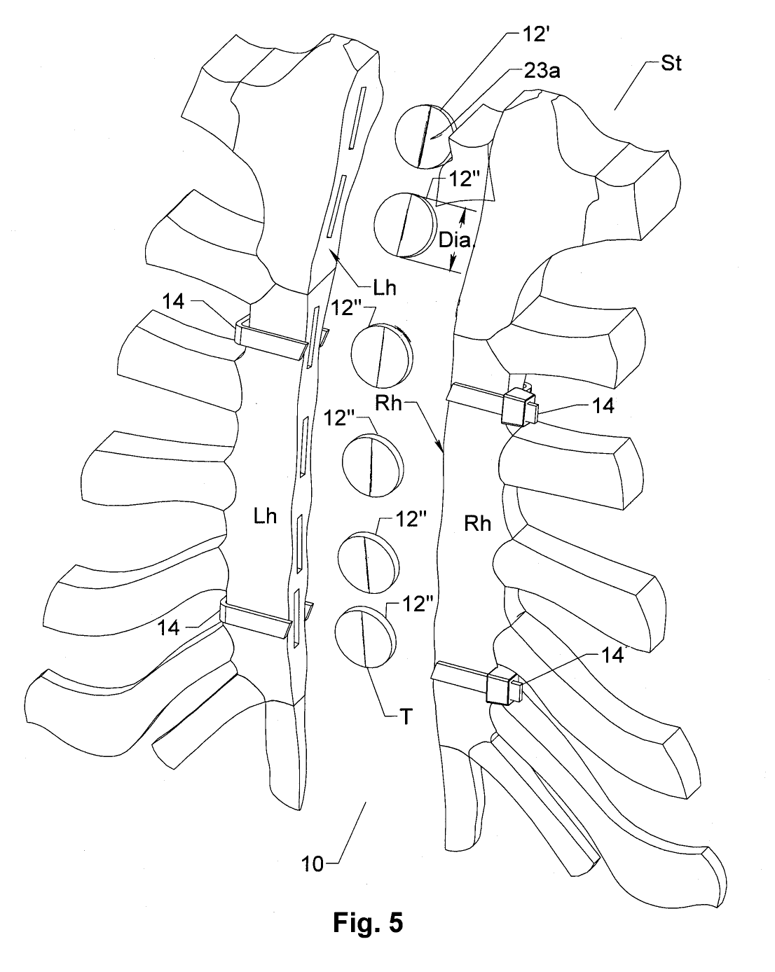

Applicant provides in embodiment 12″ (FIGS. 4 and 5) an alignment disk for insertion into walls excavated in the opposed faces, which excavated walls are designed to snugly receive alignment disk 12″ during the closure operation, such that, upon closure (see FIG. 4) the disks are substantially or fully enclosed and the sternum faces are joined. Alignment disks may have central line marking 23a to help the surgeon in placing the disks centered in the holes. Alignment disks may have a diameter Dia and a thickness T, the dimensions of which may vary and would be selected to be suitable to the size and age of the patient.

Alignment pin 12′″ (see FIG. 3A) is seen to generally conform to the dimensions of alignment pin 12′, but contains a skeletal (partial open) body 20a from which discrete barbs 26a/28a extend therefrom, which discrete barb segments may be canted inward and flexible or elastomeric to provide the “one-way fit” of the transverse alignment pin or member.

FIG. 3B illustrates a smooth (unbarbed) embodiment 12″″ of the alignment pin. No barbs are used. The smooth embodiment may be solid (as illustrated) or skeletal with a partially opened core and channels with openings to the outer cylindrical wall (see FIG. 3C).

Applicant is seen to provide external compression members 14 for assisting in the final few millimeters of closure and for assisting in maintaining compressive forces along the opposed sternum faces to assist in the healing of the bone while the alignment members assist in preventing planar or slip displacement of one sternum half with respect to the other during the final closure of the sternum and during the post-operation healing phase.

Element 14 illustrates generally a suitably shaped compression member for engagement with the two sternum halves, typically at the intercostal spaces. Compression member 14 (see FIG. 7) may be referred to as “tie wrap clamp” and is similar, but not identical, to the Synthes® GmbH Sternal ZipFix System available from Synthes GmbH, Germany. The tie wrap clamp embodiment 14 is typically configured with body 32, locking head 34, and an end 36, which may be clipped after closure and clamping. Body 32 may have a smooth surface on one side and a toothed surface (shown in FIG. 7); the toothed surface engaging tooth elements in locking head 34, so that the body may be pulled through the locking head in one direction but cannot back out. This is similar to the action of “zip” ties. Applicant's tie wrap clamp may be used as an external compression member 14 to help ease the left and right halves of the sternum together with the internal alignment members in place and/or maintain them under proper compression during the healing phase. The compression members are typically flexible and may also be bio-absorbable.

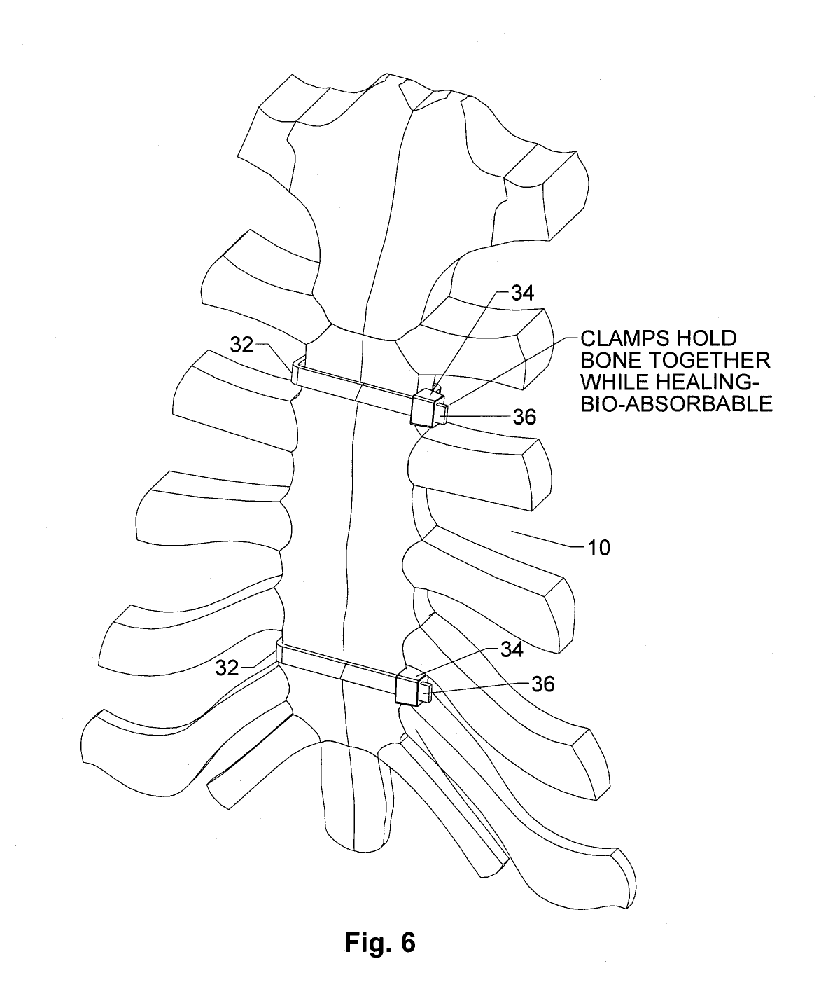

In Applicant's modified sternotomy, the sternum is cut along a vertical axis by a saw in ways known in the art. It is then broken apart and separated. Following the operation, for example, a heart transplant, multiple paired holes are excavated by the surgeon at pre-marked locations CPM (see FIGS. 4 and 6). That is to say, the surgeon laterally marks the bone perpendicular to the transverse cut, typically prior to cutting or cracking the sternum open, which CPM marks designate locations for later excavation (so the excavated holes and transverse members meet in proper alignment).

Following separation and following the operation and before closure, the CPM marks are used to excavate holes in the opposed faces dimensioned according to the shape of the alignment pin used. There may be cylindrical holes, for example, of a diameter about equal to or slightly less than the diameter of the body of pin 12′, 12′″ or 12″″ or they may be D-shaped holes for receipt of the disk assembly 12″. Typically, hole in each sternum half is about half the total length of the transverse member or disk. Any number of transverse alignment member/hole combinations may be used. They may be placed between the ribs or at any other suitable location.

The surgeon then places a set of alignment members to one side of the sternum, either left or right, and brings the two in close proximity to each other so that the other halves of the alignment members may slide into the facing holes. Either before this operation or before the alignment/placement step, any of the external compression members 14 may be used to urge the two halves close together and to bring the sternum faces substantially flush to one another with the internal alignment members therein. After the tie wrap clamp secures the two sternum halves, the excess end portion may be clipped as seen in FIG. 5. The surgeon will tie wrap to the proper compression setting, sew up the patient, and recovery will commence. With Applicant's system 10 combining (typically) fully enclosed implantable transverse alignment pins/members and tie wrap, adjustable members capable of maintaining or applying and maintaining compression to the two faces when they are brought together, a successful recovery is anticipated with a minimum of flex or movement between the sternum halves.

Any suitable number of internal alignment members (and any style or combination of embodiments) may be used. Any number of external compression members, in any suitable location, may be used. Bioabsorbable materials are preferred, but not necessary.

Although the invention has been described with reference to a specific embodiment, this description is not meant to be construed in a limiting sense. On the contrary, various modifications of the disclosed embodiments will become apparent to those skilled in the art upon reference to the description of the invention. It is therefore contemplated that the appended claims will cover such modifications, alternatives, and equivalents that fall within the true spirit and scope of the invention.

1. A sternum fixation assembly for fixing the sternum following a modified median sternotomy, which vertically divided sternum has left and right median facing walls created by the sternotomy, which include paired left and right opposing excavations, the assembly comprising:
an implantable, bio-absorbable transverse member configured for enclosure into the excavations of the left half and a right half of a vertically divided sternum; and
an elongated, coupleable, adjustable compression member dimensioned for receipt about the inter-rib spaces about the sternum and adapted to apply compression to the outer ridges of the sternum to help maintain the sternum in closure.

2. The sternum fixation assembly of claim 1, wherein the transverse member is cylindrical and includes a body and multiple extending flexible barbs.

3. The sternum fixation assembly of claim 2, wherein the barbs are inwardly canted.

4. The sternum fixation assembly of claim 2, wherein the barbs are inwardly canted and have a full perimeter.

5. The sternum fixation assembly of claim 2, wherein the barbs are inwardly canted and have multiple discrete barb members.

6. The sternum fixation assembly of claim 2, wherein the barbs have a diameter configured to engage and flex when inserted into the excavations.

7. The sternum fixation assembly of claim 2, wherein the body has a central portion.

8. The sternum fixation assembly of claim 7, wherein the central portion has indicia thereon.

9. The sternum fixation assembly of claim 2, wherein the body includes a channel therein.

10. The sternum fixation assembly of claim 1, wherein the transverse member has smooth outer cylindrical walls.

11. The sternum fixation assembly of claim 10, wherein the transverse member has at least one internal channel.

12. The sternum fixation assembly of claim 1, wherein the transverse member is disc shaped.

13. The sternum fixation assembly of claim 1, wherein the body has a central portion; and wherein the central portion has indicia thereon.

14. The sternum fixation assembly of claim 1, wherein the compression member is a zip tie.

15. The sternum fixation assembly of claim 14, wherein the zip tie is biodegradable.

16. A sternum fixation assembly for fixing the sternum following a modified median sternotomy, which vertically divided sternum has left and right median facing walls created by the sternotomy, which include paired left and right opposing excavations, the assembly comprising:
an implantable, bio-absorbable transverse member configured for enclosure into the excavations of the left half and a right half of a vertically divided sternum; and
an elongated, coupleable, adjustable compression member dimensioned for receipt about the inter-rib spaces about the sternum and adapted to apply compression to the outer ridges of the sternum to help maintain the sternum in closure;
wherein the transverse member is cylindrical and includes a body and multiple extending flexible barbs;
wherein the barbs are inwardly canted;
wherein the barbs have a diameter configured to engage and flex when inserted into the excavations;
wherein the compression member is a zip tie; and
wherein the zip tie is biodegradable.
